(19) United States
(12) Patent Application Publication
(10) Pub. No.: US 2025/0281453 A1
(43) Pub. Date: Sep. 11, 2025

(54) COMBINATION THERAPIES COMPRISING QTX125

(71) Applicant: QUIMATRYX, S.L., Guipúzcoa, San Sebastián (ES)

(72) Inventors: Yosu Ion VARA SALAZAR, Guipúzcoa, San Sebastián (ES); Eneko ALDABA AREVALO, Guipúzcoa, San Sebastián (ES); Tamara BELLO IGLESIAS, Guipúzcoa, San Sebastián (ES); Laureano SIMON BUELA, Guipúzcoa, San Sebastián (ES)

(21) Appl. No.: 18/861,550

(22) PCT Filed: May 19, 2022
(86) PCT No.: PCT/EP2022/063661

§ 371 Date: Oct. 29, 2024

(30) Foreign Application Priority Data
Apr. 29, 2022 (CN) 2022104755471

(51) Int. Cl. A61K 31/4025; A61K 31/44; A61K 31/7068; A61K 38/05; A61P 35/00

(52) U.S. Cl. CPC A61K 31/4025; A61K 31/44; A61K 31/7068; A61K 38/05; A61P 35/00

(57) ABSTRACT

The present invention relates to combination therapies, particularly for use in treatment of cancers. Disclosed herein are methods of treatment of a proliferative disorder in a mammalian subject, comprising administering a therapeutically effect amount of a compound of Formula I to a patient in need thereof, wherein the method comprises administering the compound to the subject simultaneously, sequentially or separately with a second pharmaceutical agent. Pharmaceutical compositions comprising a compound of Formula I, and methods of use thereof are also provided. The invention finds particular use in the treatment of cancers.

The application claims priority from CN202210475547.1 as filed 29 Apr. 2022, the contents and elements of which are herein incorporated by reference for all purposes.

### FIELD OF INVENTION

The present invention relates to combination therapies, particularly for use in treatment of cancers.

### BACKGROUND

### Histone Deacetylase (HDAC)

Histone deacetylases (HDAC) constitute an interesting therapeutic target for the treatment of cancer (cf. P. A. Marks et al. in Nature Rev. Cancer 2001, 1, 194; J. E. Bolden et al. in Nature Rev. Drug Discov. 2006, 5, 769; P. Gallinari et al. in Cell Res. 2007, 17, 195; K. B. Glaser in Biochem. Pharmacol. 2007, 74, 659; L. Pan et al. in Cell. Mol. Immunol. 2007, 4, 337; M. Haberland et al. in Nature Rev. Genetics 2009, 10, 32; Y. Zhang et al. in Curr. Med. Chem. 2008, 15, 2840; S. Ropero, and M. Esteller in Mol. Oncol. 2007, 1, 19) and other diseases such as those related to central nervous system, such as autoimmune diseases (cf. A. G. Kazantsev, and L. M. Thompson in Nature Rev. Drug Discov. 2006, 7, 854).

Several families of HDAC inhibitors (HDACis) have been designed, whose general structures can be found in different reviews (cf. A. Villar-Garea, and M. Esteller in Int. J. Cancer 2004, 112, 171; T. A. Miller et al. in J. Med. Chem. 2003, 46, 5097; T. Suzuki and N. Miyata in Curr. Med. Chem. 2005, 12, 2867; M. Paris et al. in J. Med. Chem. 2008, 51, 1505). The general structure of these inhibitors consists of a cyclic structure, a spacer and a chelating group capable of binding to the Zn (II) cation of the active centre of the different HDAC isoforms that belong to the class I (HDAC1, HDAC2, HDAC3 and HDAC8), class II (HDAC4, HDAC5, HDAC6, HDAC7, HDAC9 and HDAC10) and class IV (HDAC11).

The mechanism of action of the HDAC inhibitors is explained by their antagonist properties against histone deacetylases involved in the regulation of processes related to apoptosis, cell growth, tumour progression, cancer metastasis, cell adhesion and others. These properties prevent the binding of HDACs to their natural ligands, which can be histones or cytoplasmic proteins such as tubulin, as well as their normal catalytic activation, namely the deacetylation of E-N-acetyl lysine residues present in these proteins.

Despite having a similar inhibition mode, occasionally some selectivity in the inhibition of different HDAC isoforms has been observed (cf. J. C. Wong et al. in J. Am. Chem. Soc. 2003, 125, 5586; G. Estiu et al. in J. Med. Chem. 2008, 51, 2898). The mentioned selectivity is of therapeutic interest (cf. K. V. Butler and A. P. Kozikowski in Curr. Pharm. Design 2008, 14, 505; T. C. Karagiannis and A. El-Osta in Leukemia 2007, 21, 61).

### HDAC Inhibitors

One important class of HDAC inhibitors are trisubstituted pyrrolic derivatives connected with the chelating groups through aromatic and heteroaromatic groups, as described for example, in WO2011/039353. These compounds have been shown to be effective in the treatment of cancer (cf. WO 2011/039353).

In addition, there compounds have been shown to be effective in the treatment of several autoimmune diseases. For example, these compounds have been shown to be effective in animal models of autoimmune hepatitis and autoimmune encephalomyelitis (cf. WO 2018/087082).

An especially promising compound is 3-(3-Furyl)-N-{4-[(hydroxyamino) carbonyl] benzyl}-5-(4-hydroxyphenyl)-1 Hpyrrole-2-carboxamide (referred to herein as QTX125).

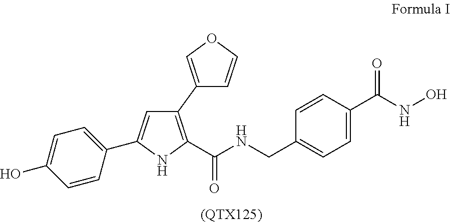

QTX125 is a highly selective and highly potent HDAC 6 inhibitor. It has shown high antitumoral efficacy in mantle cell lymphoma (cf. Perez-Salvia, M. et al in Haematologica 2018; 103: e540), lung cancer and pancreatic cancer xenograft smurine models. QTX125 has also shown high efficacy in two different multiple sclerosis mice models (cf. WO 2018/087082).

### Combination Therapies

Chemotherapy is the gold-standard of care in the treatment of several cancers, and involves the administration of an anti-cancer pharmaceutical agents to a patient in need thereof. However, the development of drug resistance and/or adverse effects is widely known to limit the therapeutic utility and clinical tolerance of chemotherapeutic agents (cf. Housman, G et al. in Cancers (Basel) 2014 6 (3): 1769-1792; Prieto-Callajero, B et al. in Medicine 2020 99 (33): pe21695).

‘Combination therapies’ utilise individually efficacious chemotherapeutic agents (having different mechanisms of action) to reduce the incidence of drug resistance, by broadening the selection pressures impressed upon individual tumour cells. Combination therapies also allow for the individual dose of each constituent chemotherapeutic agent to be reduced, thereby reducing the occurrence of adverse effects in a dose-dependent manner (Mokhtari, R. et al. in Oncotarget 2017 6 (23): 38025-38043).

However, the use of multiple pharmaceutical agents in combination may lead to drug-drug interactions, often limiting the oncolytic efficacy of the constituent pharmaceutical agents. Drug-drug interactions are especially common and complex in cancer patients, as these individuals are often the subject of polypharmacy. Drug-drug interactions of this kind limit the therapeutic utility and clinical tolerance of combination therapies.

Synergistic antitumour activity has been reported for a combination of an HDAC inhibitor, Suberoylanilide hydroxamic acid (SAHA; also known as Vorinostat), and an EGFR inhibitor, Gefitinib for head and neck cancer (c.f. Citro et al., British Journal of Cancer (2019) 120:658-667). However, the presence or absence of synergistic antitumour activity for a specific combination of agents and/or for a specific cancer type may be unpredictable. Moreover, QTX125 differs from other HDAC inhibitors such as SAHA, many of which are relatively non-specific “pan-HDAC” inhibitors, in that QTX125 has been shown to be largely HDAC6-specific. It is therefore uncertain whether or in which circumstances drug combinations including QTX125 would exhibit enhanced anti-cancer activity.

In this context, there remains a need within the art for efficacious and well-tolerated combination therapies for use in the treatment of cancer. In particular, there remains a significant unmet clinical need for novel combination therapies which achieve a synergistic effect, improving the oncolytic efficacy of the constituent pharmaceutical agents if used in combination.

### SUMMARY OF THE INVENTION

It is an object of the present invention to provide combination therapies, comprising QTX125 administered simultaneously, sequentially or separately with a second pharmaceutical agent, in order to address the problems faced by conventional chemotherapeutic regimens as outlined above.

Accordingly, a first aspect of the invention provides a method of treatment of a proliferative disorder in a mammalian subject, comprising: administering a therapeutically effective amount of a compound of formula I to a patient in need thereof,

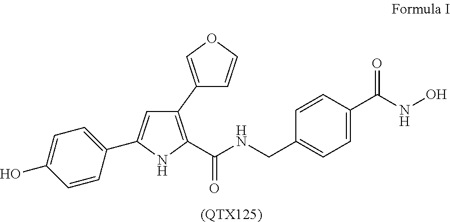

in which the method comprises administering a compound of formula I to the subject simultaneously, sequentially or separately with a second pharmaceutical agent selected from the group consisting of: (i) a protein kinase inhibitor; (ii) a ribonucleotide reductase inhibitor; and (iii) a proteasome inhibitor.

In some aspects of the invention, the proliferative disorder is a cancer, for example, a solid tumour. Solid tumours of the disclosure include colorectal tumours, pancreatic tumours, hepatic tumours and ovarian tumours.

In aspects of the invention in which the second pharmaceutical agent is a protein kinase inhibitor, the protein kinase inhibitor may be tyrosine protein kinase inhibitor. An example of such tyrosine protein kinase inhibitor is sorafenib. In such aspects, the method of treatment may be a method of treating colorectal, pancreatic, hepatic or ovarian cancer. For example, the method of treatment may be a method of treating colorectal or hepatic cancer.

In aspects of the invention in which the second pharmaceutical agent is a ribonucleotide reductase inhibitor, the ribonucleotide reductase inhibitor may be gemcitabine. In such aspects, the method of treatment may be a method of treating pancreatic, hepatic or ovarian cancer. For example, the method of treatment may be a method of treating pancreatic or ovarian cancer.

In aspects of the invention in which the second pharmaceutical agent is a proteasome inhibitor, the proteasome inhibitor may be a 26S proteasome inhibitor. An example of such 26S proteasome inhibitor is bortezomib. In such aspects, the method of treatment may be a method of treating colorectal, pancreatic, hepatic or ovarian cancer. For example, the method of treatment may be a method of treating pancreatic, hepatic or ovarian cancer. Specifically, the method may be a method of treating a hepatic or an ovarian cancer.

A second aspect of the invention provides a compound of formula I (QTX125) or a pharmaceutically acceptable salt thereof, for use in a method of treatment of a proliferative disorder in a mammalian subject in accordance with the first aspect of the invention.

A third aspect of the invention provides a pharmaceutical composition for use in a method of treatment of a proliferative disorder in a mammalian subject in accordance with the first aspect of the invention, in which said pharmaceutical composition comprises a compound of formula I (QTX125) or a pharmaceutically acceptable salt thereof, and a pharmaceutically acceptable excipient.

A fourth aspect of the invention provides second pharmaceutical agents for use in a method of treatment of a proliferative disorder in a mammalian subject in accordance with the first aspect of the invention.

For example, in some aspects of the invention, sorafenib is provided for use in a method of treatment of a proliferative disorder in a mammalian subject in accordance with the first aspect of the invention.

In other aspects, gemcitabine is provided for use in a method of treatment of a proliferative disorder in a mammalian subject in accordance with the first aspect of the invention.

In further aspects, bortezomib is provided for use in a method of treatment of a proliferative disorder in a mammalian subject in accordance with the first aspect of the invention.

A fifth aspect of the invention provides a pharmaceutical composition comprising a first pharmaceutical agent comprising a compound of formula I (QTX125) or a pharmaceutically acceptable salt thereof; a second pharmaceutical agent selected from the group consisting of: (i) a protein kinase inhibitor; (ii) a ribonucleotide reductase inhibitor; and (iii) a proteasome inhibitor; and a pharmaceutically acceptable carrier, excipient or diluent.

In some aspects of the invention, the second pharmaceutical agent is selected from the group consisting of: sorafenib, gemcitabine and bortezomib.

In further aspects, the molar ratio of the first pharmaceutical agent to the second pharmaceutical agent in pharmaceutical compositions of the fifth aspect of the invention is in the range 1:10 to 10:1.

Pharmaceutical compositions of the fifth aspect of the invention are provided herein for use in medicine, for example, for use in a method of treatment of a proliferative disorder in a mammalian subject in accordance with the first aspect of the invention. Pharmaceutical compositions of the fifth aspect of the invention may also find use in the preparation of a medicament, such as a medicament for use in a method of treatment of a proliferative disorder in a mammalian subject in accordance with the first aspect of the invention.

A sixth aspect of the invention provides the use of a compound of formula I (QTX125) or a pharmaceutically acceptable salt thereof in the preparation of a medicament. For example, a medicament for use in a method of treatment of a proliferative disorder in a mammalian subject in accordance with the first aspect of the invention.

Accordingly, a seventh aspect of the invention provides second pharmaceutical agents of the invention for use in the preparation of a medicament.

In some aspects, the invention provides the use of sorafenib in the preparation of a medicament; for example a medicament for use in a method of treatment of a proliferative disorder in a mammalian subject in accordance with the first aspect of the invention.

In other aspects, the invention provides the use of gemcitabine in the preparation of a medicament; for example a medicament for use in a method of treatment of a proliferative disorder in a mammalian subject in accordance with the first aspect of the invention.

In further aspects, the invention provides the use of bortezomib in the preparation of a medicament; for example, a medicament for use in a method of treatment of a proliferative disorder in a mammalian subject in accordance with the first aspect of the invention.

The invention referred to herein is defined by the claims.

### DESCRIPTION OF THE FIGURES

Embodiments and experiments illustrating the principles of the invention will now be discussed with reference to the accompanying figures:

FIG. 1. Dose-response curves demonstrating the oncolytic efficacy of QTX125/sorafenib combination therapy in HCT-116 colonic cancer cells.

FIG. 2. Dose-response curves demonstrating the oncolytic efficacy of QTX125/gemcitabine combination therapy in HCT-116 colonic cancer cells.

FIG. 3. Dose-response curves demonstrating the oncolytic efficacy of QTX125/bortezomib combination therapy in HCT-116 colonic cancer cells.

FIG. 4. Dose-response curves demonstrating the oncolytic efficacy of QTX125/sorafenib combination therapy in MiaPaCa-2 pancreatic cancer cells.

FIG. 5. Dose-response curves demonstrating the oncolytic efficacy of QTX125/gemcitabine combination therapy in MiaPaCa-2 pancreatic cancer cells.

FIG. 6. Dose-response curves demonstrating the oncolytic efficacy of QTX125/bortezomib combination therapy in MiaPaCa-2 pancreatic cancer cells.

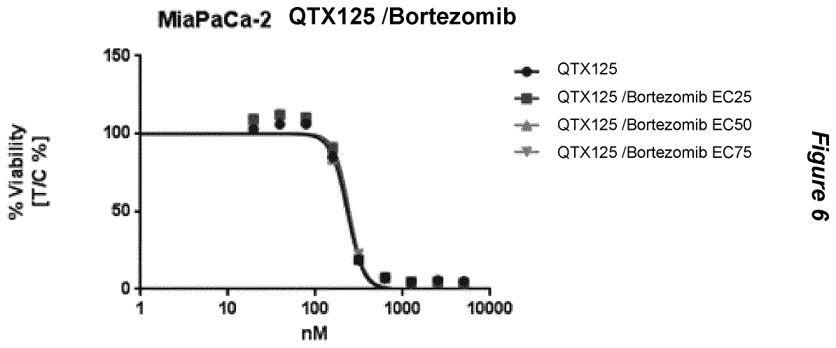

FIG. 7. Dose-response curves demonstrating the oncolytic efficacy of QTX125/sorafenib combination therapy in Hep-G2 hepatic cancer cells.

FIG. 8. Dose-response curves demonstrating the oncolytic efficacy of QTX125/gemcitabine combination therapy in Hep-G2 hepatic cancer cells.

FIG. 9. Dose-response curves demonstrating the oncolytic efficacy of QTX125/bortezomib combination therapy in Hep-G2 hepatic cancer cells.

FIG. 10. Dose-response curves demonstrating the oncolytic efficacy of QTX125/sorafenib combination therapy in SK-Ov-3 ovarian cancer cells.

FIG. 11. Dose-response curves demonstrating the oncolytic efficacy of QTX125/gemcitabine combination therapy in SK-Ov-3 ovarian cancer cells.

FIG. 12. Dose-response curves demonstrating the oncolytic efficacy of QTX125/bortezomib combination therapy in SK-Ov-3 ovarian cancer cells.

FIG. 13. Dose-response curves demonstrating the oncolytic efficacy of QTX125/sorafenib combination therapy in Panc-1 pancreatic cancer cells.

FIG. 14. Dose-response curves demonstrating the oncolytic efficacy of QTX125/gemcitabine combination therapy in Panc-1 pancreatic cancer cells.

FIG. 15. Dose-response curves demonstrating the oncolytic efficacy of QTX125/bortezomib combination therapy in Panc-1 pancreatic cancer cells.

### DETAILED DESCRIPTION

Aspects and embodiments of the present invention will now be discussed with reference to the accompanying figures. Further aspects and embodiments will be apparent to those skilled in the art. All documents mentioned in this text are incorporated herein by reference.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “about” preceding a stated value indicates that the value may have an uncertainty of ±20%, preferably ±10%, ±5%, ±2%, ±1% of the stated value. The term “room temperature” refers to the ambient temperature of a typical laboratory, which is typically between 20° C. and 30° C., preferably around 25° C., at atmospheric pressure.

The term “injection” refers to any form of injection known to a skilled person in the art such as subcutaneous, intradermal, intramuscular, intravenous, intraarterial, intracardiac, intrathecal, intraspinal, intracapsular, subcapsular, intra-orbital, intraperitoneal, intratracheal, subcuticular intraarticular, subarachnoid, and intra-sternal. Injection may refer to an infusion process (e.g. sustained administration) as well as bolus (discreate) administration.

The term “treatment” or “treating” refers to administration of a compound or a pharmaceutical composition of the invention to improve or eliminate the disease or one or more symptoms associated with the disease. The term “prevention” or “prevent” includes reducing the risk of the disease appearing or developing.

If not indicated otherwise “%” refers to percentage weight.

### Advantages

Combination therapies of the invention are highly advantageous.

For example, the QTX125-containing combination therapies described herein display a synergistic effect in mediating the efficient killing of cancer cells. This means that QTX125-containing combination therapies are highly efficacious oncolytic agents, and therefore that QTX125-containing combination therapies are attractive therapeutic tools for use in the treatment of proliferative disorders in mammalian subjects in need thereof.

Combination synergy is advantageous in that the dose of each constituent agent in the combination can be reduced, therefore overcoming the development of adverse effects that are often associated with use of individual chemotherapeutic agents at higher doses. Synergistic combination therapies are also advantageous by way of reducing the incidence of drug resistance, as the selection pressures impressed upon individual tumour cells are broadened.

### QTX125

As aforementioned, QTX125 is 3-(3-Furyl)-N-{4-[(hydroxyamino) carbonyl] benzyl}-5-(4-hydroxy phenyl)-1H-pyrrole-2-carboxamide, and has the following chemical formula:

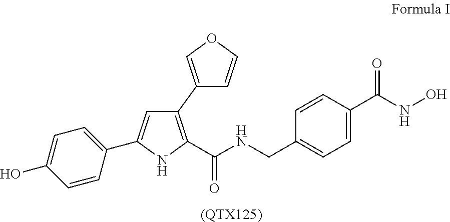

References to the term ‘QTX125’ herein are intended to include crystalline forms of QTX125, and adducts thereof. Pharmaceutically acceptable salts of compounds of formula I are also provided. Methods of preparing a compound of formula I and evidence of its biological activity for application in various medical treatments are described in e.g. WO 2011/039353 and WO 2018/087082, the contents of which are incorporated herein by reference. Advantageous pharmaceutical formulations comprising QTX125 are further described in CN 202210325433.9, the contents of which are incorporated herein by reference.

The present inventors have found that QTX125, unlike other histone deacetylase inhibitors, advantageously show no evidence of genotoxicity, in particular of clastogenicity or aneugenicity. Similarly, it has unexpectedly been observed that QTX125 possess improved pharmacokinetic properties, in particular higher half-lives and distribution volumes, than other histone deacetylase inhibitors.

The term “pharmaceutically acceptable salts” refers to salts which, when administered to the recipient, can provide (directly or indirectly) a compound as described in the present document. “Pharmaceutically acceptable” preferably refers to compositions and molecular entities that are physiologically tolerable and do not usually produce an allergic reaction or a similar unfavourable reaction such as gastric disorders, dizziness, and suchlike, when administered to a human or animal. Preferably, the term “pharmaceutically acceptable” means it is approved by a regulatory agency of a state or federal government or is included in the U.S. Pharmacopoeia or other generally recognized pharmacopoeia for use in animals, and more particularly in humans.

The preparation of salts can be accomplished by methods known in the art. For example, pharmaceutically acceptable salts may be synthesized from the original compound, which contains basic residues, by conventional chemical methods. Generally, such salts are prepared, for example, by reacting free base forms of the compound with the appropriate base or acid in water or in an organic solvent or in a mixture of both. In general, non-aqueous media like ether, ethyl acetate, ethanol, isopropanol or acetonitrile are preferred. Examples of acid addition salts include mineral acid addition salts such as, e.g. hydrochloride, hydrobromide, hydroiodide, sulfate, nitrate, phosphate salts and organic acid addition salts such as, for example, acetate, maleate, fumarate, citrate, oxalate, succinate, tartrate, malate, mandelate, methanesulfonate and p-toluenesulfonate salts. Examples of base addition salts include inorganic salts such as, for example, sodium, potassium, calcium, ammonium, magnesium, aluminum and lithium salts, and organic salts such as, for example, ethylenediamine, ethanolamine, N,N-dialkylenethanolamine, triethanolamine, glucamine and basic salts of amino acids.

### Proliferative Disorders and Cancers

A method of treatment of a proliferative disorder in a mammalian subject is provided herein, in which said method comprises administering a compound of Formula I to the subject simultaneously, sequentially or separately with a second pharmaceutical agent selected from the group consisting of: (i) a protein kinase inhibitor; (ii) a ribonucleotide reductase inhibitor; and (iii) a proteasome inhibitor.

In some aspects of the disclosure, the proliferative disorder is a cancer. The cancer may comprise a solid tumour, for example, a colorectal tumour, a fibrosarcoma, a gastric tumour, a glioblastoma, a renal tumour, a hepatic tumour, a pulmonary tumour, a melanoma, a nasopharyngeal tumour, an oral tumour, an osteosarcoma, an ovarian tumour, a pancreatic tumour or a prostatic tumour. Preferably, the cancer may comprise a colorectal, a pancreatic, a hepatic or an ovarian tumour. Alternatively, the cancer may comprise a blood cancer, such as a lymphoma, a leukaemia, or a myeloma.

### Second Pharmaceutical Agents

Provided herein are methods of treatment, methods of use and pharmaceutical compositions which utilise compounds of formula I (or pharmaceutically acceptable salts thereof) alongside a second pharmaceutical agent selected from the list consisting of: (i) protein kinase inhibitors; (ii) ribonucleotide reductase inhibitors and (iii) proteasome inhibitors.

Protein kinase inhibitors are biologically active agents which inhibit the action of one or more protein kinases. Protein kinases are enzymes which act to add a phosphate group (PO₄) to a protein, and which act to modify the function of proteins and signalling pathways.

Protein kinase inhibitors suitable for use in the disclosure may include: adavosertib, afatinib, axitinib, bosutinib, cetuximab, cobimetinib, crizotinib, cabozantinib, dacomitinib, dasatinib, entrectinib, erdafitinib, erlotinib, fostamatinib, gefitinib, ibrutinib, imatinib, lapatinib, lenvatinib, mubritinib, nilotinib, pazopanib, pegaptanib, ruxolitinib, sorafenib, sunitinib, SU6656, tucatinib, vandetanib and vemurafenib.

In preferred aspects of the disclosure, the protein kinase inhibitor is a tyrosine protein kinase inhibitor. In the most preferred aspects, the protein kinase inhibitor is sorafenib.

Ribonucleotide reductase inhibitors are biologically active agents which prevent the enzymatic activity of ribonucleotide reductase (RNR), also known as ribonucleoside diphosphate reductase (rNDP). In other words, ribonucleotide reductase inhibitors inhibit the formation of deoxyribonucleotides (a component of DNA) from ribonucleotides.

Ribonucleotide reductase inhibitors suitable for use in the disclosure may include: motexafin gadolinium, hydroxyurea, fludarabine, cladribine, gemcitabine, tezacitabine, triapine, gallium maltolate, and gallium nitrate.

In preferred aspects of the disclosure, the ribonucleotide reductase inhibitor is gemcitabine. The proteasome is a protease complex which degrades damaged or unneeded proteins by way of proteolysis. The 26S proteasome (comprising one 20S protein subunit and two 19S regulatory cap subunits) is the major protease in eukaryotic cells, and is responsible for protein degradation in both the cytosol and the nucleus. Proteasome inhibitors are biologically active agents which inhibit the action of proteasome complexes.

Proteasome inhibitors suitable for use in the disclosure may include: lactacystin, disulfiram, epigallocatechin-3-gallate, marizomib, oprozomib, delanzomib, epoxomicin, MG132, beta-hydroxy beta-methylbutyrate, bortezomib, carfilzomib and ixazomib.

In preferred aspects of the disclosure, the proteasome inhibitor is an inhibitor of the 26S proteasome. In the most preferred aspects, the protein kinase inhibitor is bortezomib.

### Methods of Treatment

The invention provides combination therapies comprising QTX125 and a second pharmaceutical agent.

In some aspects of the disclosure, a method of treatment of a proliferative disorder in a mammalian subject is provided, comprising administering a therapeutically effective amount of a compound of formula I to a patient in need thereof,

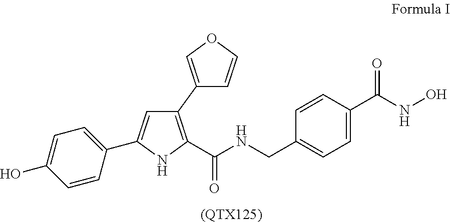

in which the method comprises administering a compound of formula I to the subject simultaneously, sequentially or separately with a second pharmaceutical agent selected from the group consisting of: (i) a protein kinase inhibitor; (ii) a ribonucleotide reductase inhibitor; and (iii) a proteasome inhibitor.

The methods disclosed herein may be methods of treating cancer in a mammalian subject. Such methods may be methods of treating a solid tumour in subject in need thereof. Solid tumours suitable for treatment by way of methods of the invention may include, for example, a colorectal, pancreatic, hepatic or an ovarian cancer. In some embodiments in accordance with any aspect of the present invention, the colorectal cancer may be a colon cancer.

In preferred methods of the invention, the protein kinase inhibitor is sorafenib.

In other preferred methods of the invention, the ribonucleotide reductase inhibitor is gemcitabine.

In further preferred methods of the invention, the proteasome inhibitor is bortezomib.

Methods which Utilise Protein Kinase Inhibitors

Some aspects of the invention provide methods of treatment of a proliferative disorder in a mammalian subject, said methods comprising: administering to a patient in need thereof a therapeutically effective amount of a compound of formula I, or pharmaceutically acceptable salt thereof, simultaneously, sequentially or separately with a protein kinase inhibitor.

For example, the invention provides methods of treatment of cancer (including of solid tumours) in mammalian subjects in need thereof, by way of administering to said subject a therapeutically effective amount of a compound of formula I, or pharmaceutically acceptable salt thereof, simultaneously, sequentially or separately with a protein kinase inhibitor.

In preferred aspects of such methods, the solid tumour may be a colorectal, pancreatic, hepatic or an ovarian tumour. More preferably in such methods, the solid tumour may be a colorectal or a hepatic tumour.

For example, specific aspects of the invention provide methods of treating colorectal cancer in a mammalian subject in need thereof, comprising administering to said subject a therapeutically effective amount of a compound of formula I, or pharmaceutically acceptable salt thereof, simultaneously, sequentially or separately with a protein kinase inhibitor.

Also provided are methods of treating pancreatic cancer in a mammalian subject in need thereof, comprising administering to said subject a therapeutically effective amount of a compound of formula I, or pharmaceutically acceptable salt thereof, simultaneously, sequentially or separately with a protein kinase inhibitor.

Provided herein are methods of treating hepatic cancer in a mammalian subject in need thereof, comprising administering to said subject a therapeutically effective amount of a compound of formula I, or pharmaceutically acceptable salt thereof, simultaneously, sequentially or separately with a protein kinase inhibitor.

Further provided are methods of treating ovarian cancer in a mammalian subject in need thereof, comprising administering to said subject a therapeutically effective amount of a compound of formula I, or pharmaceutically acceptable salt thereof, simultaneously, sequentially or separately with a protein kinase inhibitor.

Preferably, the protein kinase inhibitors suitable for use in such methods are tyrosine protein kinase inhibitors. Most preferably, the protein kinase inhibitor used in such methods is sorafenib.

Methods which Utilise Ribonucleotide Reductase Inhibitors

Some aspects of the invention provide methods of treatment of a proliferative disorder in a mammalian subject, said methods comprising: administering to a patient in need thereof a therapeutically effective amount of a compound of formula I, or pharmaceutically acceptable salt thereof, simultaneously, sequentially or separately with a ribonucleotide reductase inhibitor.

For example, the invention provides methods of treatment of cancer, including of solid tumours, in mammalian subjects in need thereof, by way of administering to said subject a therapeutically effective amount of a compound of formula I, or pharmaceutically acceptable salt thereof, simultaneously, sequentially or separately with a ribonucleotide reductase inhibitor.

In preferred aspects of such methods, the solid tumour may be a colorectal, pancreatic, hepatic or an ovarian tumour. More preferably, the solid tumour may be a pancreatic, hepatic or an ovarian tumour. Most preferably, the solid tumour may be a pancreatic or an ovarian tumour. Specific aspects of the invention provide methods of treating colorectal cancer in a mammalian subject in need thereof, comprising administering to said subject a therapeutically effective amount of a compound of formula I, or pharmaceutically acceptable salt thereof, simultaneously, sequentially or separately with a ribonucleotide reductase inhibitor.

Also provided herein are methods of treating pancreatic cancer in a mammalian subject in need thereof, comprising administering to said subject a therapeutically effective amount of a compound of formula I, or pharmaceutically acceptable salt thereof, simultaneously, sequentially or separately with a ribonucleotide reductase inhibitor.

Provided herein are methods of treating hepatic cancer in a mammalian subject in need thereof, comprising administering to said subject a therapeutically effective amount of a compound of formula I, or pharmaceutically acceptable salt thereof, simultaneously, sequentially or separately with a ribonucleotide reductase inhibitor.

Further provided are methods of treating ovarian cancer in a mammalian subject in need thereof, comprising administering to said subject a therapeutically effective amount of a compound of formula I, or pharmaceutically acceptable salt thereof, simultaneously, sequentially or separately with a ribonucleotide reductase inhibitor.

Preferably, the ribonucleotide reductase inhibitor used in such methods is gemcitabine.

Methods which Utilise Proteasome Inhibitors

Some aspects of the invention provide methods of treatment of a proliferative disorder in a mammalian subject, said methods comprising: administering to a patient in need thereof a therapeutically effective amount of a compound of formula I, or pharmaceutically acceptable salt thereof, simultaneously, sequentially or separately with a proteasome inhibitor.

For example, the invention provides methods of treatment of cancer, including of solid tumours, in mammalian subjects in need thereof, by way of administering to said subject a therapeutically effective amount of a compound of formula I, or pharmaceutically acceptable salt thereof, simultaneously, sequentially or separately with a proteasome inhibitor.

In preferred aspects of such methods, the solid tumour may be a colorectal, pancreatic, hepatic or an ovarian tumour. More preferably, the solid tumour may be a pancreatic, hepatic or ovarian tumour. Most preferably, the solid tumour may be a hepatic or an ovarian tumour.

Specific aspects of the invention provide methods of treating colorectal cancer in a mammalian subject in need thereof, comprising administering to said subject a therapeutically effective amount of a compound of formula I, or pharmaceutically acceptable salt thereof, simultaneously, sequentially or separately with a proteasome inhibitor.

Also provided herein are methods of treating pancreatic cancer in a mammalian subject in need thereof, comprising administering to said subject a therapeutically effective amount of a compound of formula I, or pharmaceutically acceptable salt thereof, simultaneously, sequentially or separately with a proteasome inhibitor.

Provided herein are methods of treating hepatic cancer in a mammalian subject in need thereof, comprising administering to said subject a therapeutically effective amount of a compound of formula I, or pharmaceutically acceptable salt thereof, simultaneously, sequentially or separately with a proteasome inhibitor.

Further provided are methods of treating ovarian cancer in a mammalian subject in need thereof, comprising administering to said subject a therapeutically effective amount of a compound of formula I, or pharmaceutically acceptable salt thereof, simultaneously, sequentially or separately with a proteasome inhibitor.

Preferably, the proteasome inhibitors suitable for use in such methods are 26S proteasome inhibitors. Most preferably, the proteasome inhibitor used in such methods is bortezomib.

### Pharmaceutical Agents of the Invention

The invention provides a compound of formula I, for use in a method of treatment of a proliferative disorder in a mammalian subject in need thereof, said method comprising administering the compound of formula I to the subject simultaneously, sequentially or separately with a second pharmaceutical agent selected from the group consisting of: (i) a protein kinase inhibitor; (ii) a ribonucleotide reductase inhibitor; and (iii) a proteasome inhibitor.

In some aspects, the compound of formula I may be used in a method of treating a cancer in a subject. For example, a colorectal, pancreatic, hepatic, or an ovarian cancer.

In some aspects, the second pharmaceutical agent of the invention is selected from the group consisting of: sorafenib, gemcitabine and bortezomib.

Accordingly, the invention also provides sorafenib for use in a method of treating a proliferative disorder in a mammalian subject in need thereof, in which said method comprises administering sorafenib to a patient simultaneously, sequentially or separately with a compound of formula I, or a pharmaceutically acceptable salt thereof.

In preferred aspects of the invention, such methods are methods of treating cancer in a subject, for example, a colorectal, pancreatic, hepatic, or an ovarian cancer. In the most preferred aspects, the method is a method of treating a colorectal or hepatic tumour.

Similarly, the invention provides gemcitabine for use in a method of treating a proliferative disorder in a mammalian subject in need thereof, in which said method comprises administering gemcitabine to a patient simultaneously, sequentially or separately with a compound of formula I, or a pharmaceutically acceptable salt thereof.

In preferred aspects of the invention, such methods are methods of treating cancer in a subject, for example, a colorectal, pancreatic, hepatic, or an ovarian cancer. In more preferred aspects, the method is a method of treating a pancreatic, hepatic or an ovarian tumour. In the most preferred aspects, the method is a method of treating a pancreatic or a hepatic tumour.

The invention further provides bortezomib for use in a method of treating a proliferative disorder in a mammalian subject in need thereof, in which said method comprises administering bortezomib to a patient simultaneously, sequentially or separately with a compound of formula I, or a pharmaceutically acceptable salt thereof.

In preferred aspects of the invention, such methods are methods of treating cancer in a subject, for example, a colorectal, pancreatic, hepatic, or an ovarian cancer. In more preferred aspects, the method is a method of treating a pancreatic, hepatic or an ovarian tumour. In the most preferred aspects, the method is a method of treating a hepatic or an ovarian tumour.

### Methods of Use

Provided herein are compounds of formula I for use in medicine. Such use may include the administration of a compound of formula I to a subject simultaneously, sequentially or separately with a second pharmaceutical agent selected from the group consisting of: (i) a protein kinase inhibitor; (ii) a ribonucleotide reductase inhibitor; and (iii) a proteasome inhibitor. Similarly, pharmaceutical compositions comprising a compound of formula I and a pharmaceutically acceptable excipient, and optionally a second pharmaceutical agent selected from the group consisting of: (i) a protein kinase inhibitor; (ii) a ribonucleotide reductase inhibitor; and (iii) a proteasome inhibitor are provided herein for use in medicine. Such uses may include a use in the preparation of a medicament for a method of treatment. For example, a method of treating a proliferative disorder or a cancer in a mammalian subject, such as a human patient.

Furthermore, sorafenib, gemcitabine and bortezomib are provided herein, for use in the preparation of a medicament for a method of treatment. For example, a method of treating a proliferative disorder or a cancer in a mammalian subject, such as a human patient.

Subjects, Dosing and Methods of Administration

The term ‘a mammalian subject’ encompasses all mammals. A subject may therefore be a rat, mouse, feline, canine, equine, porcine, ovine, bovine, primate or human. Preferably, the subject is a human patient.

In general, the effective amount of the compound of formula I to be administered will depended on a range of factors, such as the severity of the disorder being treated and the subject's weight. The active compounds will normally be administered one or more times a day for example 1, 2, 3, or 4 times daily, with typical total daily doses in the range from 0.01 up to 1,000 mg/kg/day.

Preferably, the compound of formula I is administered to human patients at a dosage of 0.5 to 50 mg/kg, preferably from 0.5 to 30 mg/kg, preferably from 1 to 20 mg/kg, more preferably from 5 to 10 mg/kg.

Preferably, the compound of formula I is administered to human patients at a dosage of from 25 mg to 4500 mg, preferably from 50 mg to 3000 mg, preferably from 250 mg to 1500 mg per day.

Preferably, a compound of formula I, or a pharmaceutical composition comprising a compound of formula I is administered via injection. Such administration may be both via infusion (continuous) or bolus (discreate) administration. The method of administration via injection may be, for example, subcutaneous, intradermal, intramuscular, intravenous, intraarterial, intracardiac, intrathecal, intraspinal, intracapsular, subcapsular, intra-orbital, intraperitoneal, intratracheal, subcuticular, intraarticular, subarachnoid, and intra-sternal injection. Preferably, the administration is by intravenous infusion or intravenous injection (bolus administration). More preferably, the administration is by intravenous infusion.

### Pharmaceutical Compositions

Pharmaceutical compositions of the disclosure may comprise a compound of formula I and a pharmaceutically acceptable excipient. For example, pharmaceutical compositions may comprise a crystalline form of a compound of formula I, a crystalline form of an adduct of a compound of formula I, and a pharmaceutically acceptable excipient.

Exemplary final concentrations of QTX125 in pharmaceutical compositions disclosed herein are at least 8 mg/mL, optionally up to 20 mg/mL, such as 8.5 mg/mL or more, 9 mg/mL or more and more preferably 9.5 mg/ml or more.

A pharmaceutical composition according to the present invention may comprise, in addition to the compound of formula I as described herein, one or more other pharmaceutically acceptable ingredients well known to those skilled in the art, including, but not limited to: pharmaceutically acceptable carriers, diluents, excipients, adjuvants, buffers, pH modifiers, preservatives, anti-oxidants, bacteriostats, stabilisers, suspending agents, solubilisers, surfactants (e.g., wetting agents), colouring agents, and isotonicising solutes (i.e., which render the formulation isotonic with the blood, or other relevant bodily fluid, of the intended recipient). Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 18th edition, Mack Publishing Company, Easton, Pa., 1990; and Handbook of Pharmaceutical Excipients, 2nd edition, 1994.

Optionally, the pharmaceutical composition according to the present invention further comprises a buffer (i.e. the composition further comprises buffer salts dissolved therein). Optionally, the said buffer may be selected from the group of MES, Bis-Tris, ADA, ACES, PIPES, MOPSO, BES, MOPS, TES, HEPES, DIPSO, MOBS, TAPSO, Tris-HCl, HEPPSO, POPSO, TEA, EPPS, Tricine, Gly-Gly, Bicine, HEPBS, TAPS, AMPD, TABS, AMPSO, CHES, CAPSO, APS, CHAPS, CABS, Phosphate and histidine or a combination of the above. Without wishing to be bound by theory, it is believed that the use of a buffer may help to stabilise the composition at physiological pH. The concentration of the buffer salt in the aqueous pharmaceutical composition may range from 1 mM to 1 M, preferably 1 mM to 100 mM, preferably 5 mM to 50 mM, preferably 5 mM to 20 mM.

The pharmaceutical composition may also comprise counter-ions and salts, such as sodium counter ions, chloride ions or NaCl dissolved is solution.

The pharmaceutical composition may also comprise, in addition to a compound of formula I, one or more other active agents, for example, one or more other therapeutic or prophylactic agents. In some aspects, such pharmaceutical compositions may be utilised to provide a combination therapy. In particular, pharmaceutical compositions described herein may comprise a second pharmaceutical agent such as a protein kinase inhibitor such as sorafenib, a ribonucleotide reductase inhibitor such as gemcitabine, or a proteasome inhibitor such as bortezomib. The second pharmaceutical agent may be part of the same composition or may be provided as a separate composition and can be administered at the same time or at different times.

For example, disclosed herein is a pharmaceutical composition comprising a first pharmaceutical agent comprising a compound of formula I or a pharmaceutically acceptable salt thereof; a second pharmaceutical agent selected from the group consisting of: (i) a protein kinase inhibitor; (ii) a ribonucleotide reductase inhibitor; and (iii) a proteasome inhibitor; and a pharmaceutically acceptable carrier, excipient or diluent. In some aspects, the second pharmaceutical agent is selected from the group consisting of: sorafenib, gemcitabine and bortezomib.

Exemplary molar ratios of the first pharmaceutical agent (i.e., a compound of formula I) to the second pharmaceutical agent are from 1:40 to 1:2.5, preferably from 1:30 to 1:2.5, preferably from 1:25 to 1:2.5, preferably from 1:20 to 1:2.5, such as from 1:15 to 1:2.5, preferably from 1:10 to 1:2.5, preferably from 1:9 to 1:2.5, preferably from 1:8 to 1:2.5, preferably from 1:6 to 1:2.5, more preferably from 1:4.5 to 1:2.5. In some aspects of the disclosure, the molar ratio of the first pharmaceutical agent to the second pharmaceutical agent is in the range 1:10 to 10:1.

As will be appreciated by one of skill in the art, features and preferred embodiments of one aspect of the invention will also pertain to other aspects of the invention.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

The following examples are provided to illustrate the effectiveness of the invention. These examples are intended to be non-limiting.

### Example 1—Experimental Protocols

To assess the oncolytic efficacy of QTX125-containing combination therapies in vitro, colonic, pancreatic, hepatic and ovarian tumour cells were administered sorafenib, gemcitabine or bortezomib at EC₂₅, EC₅₀ and EC₇₅ doses. QTX125 was co-administered in order to produce a dose response curve. QTX125 EC₅₀ values calculated following administration of combination therapies were compared against QTX125 monotherapy EC₅₀ values, thereby indicating whether QTX125-containing combination therapies achieve a synergistic effect (decreased EC₅₀), an additive effect (no displacement) or an antagonistic effect (increased EC₅₀) as compared to QTX125 treatment alone.

### Cell Culture

Colonic (HCT-116; ECACC 91091005), pancreatic (MIAPaCa-2, ECACC 85062806 and Panc-1, ECACC 87092802), hepatic (Hep-G2, ECACC 85011430) and ovarian (SK-OV-3, ECACC 91091004) tumour cell lines were cultured following standard practices.

In brief, HCT-116, MiaPaCa-2 and Panc-1 cells were thawed in high glucose DMEM (Sigma D5796) with 10% heat-inactivated Foetal Calf Serum (FCS) (PAA, A15-101)). Hep-G2 and SK-Hep1 cells were thawed in Minimum Essential Media (MEM) (Sigma M2279) with 1% non-essential amino acids (Sigma M7145), 2 mM Glutamine (Sigma G7513) and 10% FCS. SK-OV-3 cells were thawed in McCoy's 5A medium (Sigma, M8403) with 15% FCS. All cells were passaged by washing once in cation-free dPBS (Sigma, D1408), and incubated for 3 minutes with trypsin ([0.5 μg/ml]/EDTA [0.2 μg/ml] (Sigma, T4174) in dPBS). Passage was completed at 37° C., and cells were transferred to the appropriate media. Prior to seeding at defined cell density, cells were recovered in medium, centrifuged and resuspended for counting.

### Cell Viability Assays

To prepare cells for viability assays, cells were transferred into 96 well tissue culture plates (Cultek), and resuspended in 100 UL at the following densities: 10,000 cells/well (Hep-G2 and MiaPaCa-2), 5000 cells/well (Panc-1 and SK-OV-3) or 3000 cells/well (HCT-116). Following 24 hours of incubation, media was removed and replaced with 100 UL media spiked with QTX125 and the test chemotherapeutic agents. Stock solutions for the agents used in this study are shown below in Table 1. Cells were incubated for a further 72 hours before cell viability was assessed using both ALAMAR® and hexosaminidase assays.

TABLE 1
Test agents.

| | Molecular weight | Total mass | Stock concentration |
|---|---|---|---|
| | (MW) | (mg) | (in DMSO) |
| QTX125 | 417.41 | 18.9 | 50 mM |
| Sorafenib | 464.82 | 13.2 | 50 mM |
| Gemcitabine | 263.2 | 13.3 | 50 mM |
| Bortezomib | 384.24 | 10.3 | 50 mM |

For the ALAMAR® blue assay, media containing QTX125 and the test chemotherapeutic agents was removed, and cells were stained using ALAMAR® blue (BioSource DAL 1100) for 4 hours at 37° C., following the manufacturer's instructions. Relative fluorescent intensity was measured using a Cytofluor® plate reader (Millipore) at 535/590 nm (Excitation/emission). This measure directly correlates with the number of viable cells present in each well.

For the hexosaminidase assay, media containing QTX125 and the test chemotherapeutic agents was removed, and cells were washed once with PBS. 60 μL of substrate solution (containing: 7.5 mM p-nitrophenol-N-acetyl-beta-D-glucosamide [Sigma N-9376], 0.1 M sodium citrate, pH 5.0 and 0.25% Triton X-100) was added to each well and cells were incubated at 37° C. for at least 1 hour. Incubating cells in this manner causes the substrate solution to become bright yellow, at which point 90 μL of developer solution (containing 50 mM Glycine pH 10.4 and 5 mM EDTA) was added to each well. Absorbance at 410 nm was recorded using a plate reader.

### Control Samples

A number of appropriate control samples were included. First, absorbance values measured in reagent ‘blank’ wells (containing cell culture medium and colorimetric agent only) were subtracted from ‘test’ sample-containing wells. ‘Blank’ absorbance values were routinely between 5 and 10% of the values recorded in test wells. Absorbance values recorded in test wells were then compared against wells containing (i) cells stimulated with QTX125 only; (ii) cells stimulated with sorafenib, gemcitabine or bortezomib only and/or (iii) unstimulated cells, as appropriate.

### Data Analysis

Control values were normalised to 100% and percentage (%) viability was calculated. Data were used to plot log dose-response curves using a sigmoid dose-response (variable slope) equation. EC₅₀ values were obtained using Equation 1, where ‘X’ is the logarithmic concentration; ‘Y’ is the response output and it is assumed that Y starts at bottom of the graph and reaches the top with an overall sigmoid shape.

$$Y = Bottom + ( Top - Bottom ) / ( 1 + 10 \hat{} ( ( Log {EC}_{50} - X ) * HillSlope ) ) . \qquad Equation 1$$

Combination indices (CIs) were calculated for all conditions tested, to enable the identification of whether QTX125-containing combination therapies achieve a synergistic, additive or antagonistic effect, as compared to QTX125 monotherapy. Cls were obtained using Equation 2, where (Dm) 1 is the ECₓ concentration of QTX125 and (D) 1 is the ECₓ concentration of QTX125 in the presence of sorafenib, gemcitabine or bortezomib.

$$Combination index ( CI ) = {( D )}_{1} / {( Dm )}_{1} \qquad Equation 2$$

### Example 2—Assessing the Oncolytic Efficacy of Individual Chemotherapeutic Agents

Prior to assessing the oncolytic efficacy of QTX125-containing combination therapies, the oncolytic effect of each test agent was assessed in isolation. Dose-response curves were produced by serial dilution (1:1), thereby allowing monotherapy EC₅₀ values to be calculated.

Chemotherapeutic agents were added to wells at a starting concentration of 100 μM. However, it was quickly determined that it was not possible to use this range for all chemotherapeutic agents tested. An optimization study was therefore completed to identify suitable maximum (‘high’) doses for each agent and in each individual cell line. The results of the optimisation study are provided in Table 2.

TABLE 2
Optimised maximum doses of test chemotherapeutic agents.
Values provided are concentrations in micromolar (μM).

| | QTX125 | Sorafenib | Gemcitabine | Bortezomib |
|---|---|---|---|---|
| HCT-116 | 1 | 100 | 10 | 1 |
| Hep-G2 | 5 | 100 | 100 | 1 |
| MiaPaCa-2 | 100 | 100 | 100 | 1 |
| Panc-1 | 100 | 100 | 100 | 1 |
| SK-OV-3 | 10 | 100 | 10 | 1 |

Only Sorafenib monotherapy facilitated the target concentration of 100 μM to be used for all cell lines. Bortezomib was observed as having the highest efficacy, as the appropriate high-dose starting concentration was determined to be as low as 1 μM. A dose of 1 UM was also determined to be a suitable high-dose for QTX125 monotherapy in HCT-116 cells. For Gemcitabine, there were variable results due to the very high sensitivity of the HCT-116 line, as compared to the relative resistance of other lines tested (Hep-G2, MiaPaCa-2, Panc-1, and SK-OV-3). The percentage of viable cells remaining in each well following exposure to high-dose chemotherapeutic agents, is shown in Tables 3.1 to 3.4, below.

TABLE 3.1
The oncolytic efficacy of high-dose QTX125 monotherapy.
Values provided are percentage (%) cell viabilities,
following 72 hours of treatment.

| | ALAMAR ® blue | Hexosaminidase |
|---|---|---|
| HCT-116 | 0.0 | 7.3 |
| Hep-G2 | 0.0 | 2.1 |
| MiaPaCa-2 | 0.0 | 7.3 |
| Panc-1 | 0.0 | 3.9 |
| SK-OV-3 | 0.0 | 5.0 |

As measured using the ALAMAR® blue assay, high-dose QTX125 monotherapy successfully mediates the killing of all cell lines tested. As measured using the hexosaminidase assay, high-dose QTX125 monotherapy was most successful as an anti-hepatic cancer agent, reducing the population of viable Hep-G2 cells by almost 98% in 72 hours.

TABLE 3.2
The oncolytic efficacy of high-dose sorafenib monotherapy.
Values provided are percentage (%) cell viabilities,
following 72 hours of treatment.

| | ALAMAR ® blue | Hexosaminidase |
|---|---|---|
| HCT-116 | 0.0 | 2.4 |
| Hep-G2 | 0.0 | 1.8 |
| MiaPaa-2 | 0.0 | 4.7 |
| Panc-1 | 0.0 | 4.4 |
| SK-OV-3 | 0.0 | 2.3 |

As measured using the ALAMAR® blue assay, high-dose sorafenib monotherapy successfully mediates the killing of all cell lines tested. As measured using the hexosaminidase assay, high-dose sorafenib was most successful as an anti-hepatic cancer agent, reducing the population of viable Hep-G2 cells by more than 98% in 72 hours.

TABLE 3.3
The oncolytic efficacy of high-dose gemcitabine
monotherapy. Values are percentage (%) cell
viabilities, following 72 hours of treatment.

| | ALAMAR ® blue | Hexosaminidase |
|---|---|---|
| HCT-116 | 1.9 | 20 |
| Hep-G2 | 24 | 54 |
| MiaPaCa-2 | 24 | 47 |
| Panc-1 | 8.5 | 62 |
| SK-OV-3 | 38 | 50 |

As measured using both the ALAMAR® blue and hexosaminidase assays, high-dose gemcitabine was most successful as an anti-colon cancer agent, reducing the population of viable HCT-116 cells by more than 98% and 80%, respectively, over the course of 72 hours.

TABLE 3.4
The oncolytic efficacy of high-dose bortezomib
monotherapy. Values are percentage (%) cell
viabilities, following 72 hours of treatment.

| | ALAMAR ® blue | Hexosaminidase |
|---|---|---|
| HCT-116 | 0.0 | 4.9 |
| Hep-G2 | 0.0 | 8.8 |
| MiaPaCa-2 | 0.0 | 4.1 |
| Panc-1 | 0.0 | 3.0 |
| SK-OV-3 | 0.0 | 9.3 |

As measured using the ALAMAR® blue assay, high-dose bortezomib monotherapy successfully mediates the killing of all cell lines tested. As measured using the hexosaminidase assay, high-dose bortezomib was most successful as an anti-pancreatic cancer agent, reducing the population of viable Panc-1 cells by 97% in 72 hours.

Cell viability data were the used to calculate EC₅₀ values for high-dose QTX125, sorafenib, gemcitabine or bortezomib monotherapy, as shown in Tables 3.5 to 3.8, below.

TABLE 3.5
Calculation of the EC₅₀ dose of QTX125 monotherapy. Values
provided are concentrations in micromolar (μM).

| | ALAMAR@ blue | Hexosaminidase |
|---|---|---|
| HCT-116 | 0.33 ± 0.03 | 0.43 ± 0.06 |
| Hep-G2 | 0.91 ± 0.20 | 3.30 ± 0.40 |
| MiaPaCa-2 | 1.79 ± 0.05 | 1.50 ± 0.20 |
| Panc-1 | 1.40 ± 0.15 | 3.60 ± 0.50 |
| SK-OV-3 | 3.00 ± 0.40 | 3.60 ± 0.40 |

As measured using the ALAMAR® blue assay, high-dose QTX125 monotherapy was most efficient at killing HCT-116 pancreatic cancer cells (having an EC₅₀ of 0.33+0.03 UM). This was corroborated through use of the hexosaminidase assay, which also identified QTX125 to be most efficient at killing HCT-116 cells (having an EC₅₀ of 0.43+0.06 UM).

TABLE 3.6
Calculation of the EC₅₀ dose of sorafenib monotherapy.
Values provided are concentrations in micromolar (μM).

| | ALAMAR@ blue | Hexosaminidase |
|---|---|---|
| HCT-116 | 04.00 ± 00.60 | 03.60 ± 00.50 |
| Hep-G2 | 00.62 ± 00.16 | 02.70 ± 00.80 |
| MiaPaCa-2 | 08.30 ± 00.70 | 06.80 ± 00.90 |
| Panc-1 | 07.20 ± 00.80 | 07.40 ± 01.00 |
| SK-OV-3 | 17.00 ± 02.80 | 12.70 ± 01.70 |

As measured using the ALAMAR® blue assay, high-dose sorafenib monotherapy was most efficient if used as an anti-pancreatic cancer agent, killing MiaPaCa-2 cells with an EC₅₀ of 0.62±0.16 μM. As measured using the hexosaminidase assay, high-dose sorafenib monotherapy was most efficient if used as an anti-hepatic cancer agent, killing Hep-G2 cells with an EC₅₀ of 2.70+0.80 μM.

TABLE 3.7
Calculation of the EC₅₀ dose of gemcitabine monotherapy.
Values provided are concentrations in micromolar (μM).

| | ALAMAR ® blue | Hexosaminidase |
|---|---|---|
| HCT-116 | — | — |
| Hep-G2 | — | — |
| MiaPaCa-2 | 0.026 ± 0.007 | 0.033 ± 0.004 |
| Panc-1 | — | — |
| SK-OV-3 | — | — |

A number of cell lines (HCT-116, Hep-G2, Panc-1 and SK-OV-3 cells) were identified as resistant to high-dose gemcitabine monotherapy, preventing the calculation of a full complement of EC₅₀ values. As identified using both the ALAMAR® blue and the hexosaminidase assay, gemcitabine was identified as a highly efficient anti-pancreatic cancer agent, killing MiaPaCa-2 cells at very low doses (having an EC₅₀ of 0.026+0.007 UM and 0.033+0.004 UM, respectively).

TABLE 3.8
Calculation of the EC₅₀ dose of bortezomib monotherapy.
Values provided are concentrations in micromolar (μM).

| | ALAMAR ® blue | Hexosaminidase |
|---|---|---|
| HCT-116 | 0.0079 ± 0.0004 | 0.0066 ± 0.0005 |
| Hep-G2 | 0.0160 ± 0.0007 | 0.0160 ± 0.0010 |
| MiaPaCa-2 | 0.0130 ± 0.0005 | 0.0086 ± 0.0005 |
| Panc-1 | 0.0062 ± 0.0010 | 0.0093 ± 0.0008 |
| SK-OV-3 | 0.0070 ± 0.0010 | 0.0068 ± 0.0004 |

As identified using the ALAMAR® blue assay, bortezomib was most efficient at killing Panc-1 pancreatic cancer cells, having an EC₅₀ of 0.0062+0.0010 UM. As identified using the hexosaminidase assay, bortezomib was most efficient at killing HCT-116 colonic cancer cells, having an EC₅₀ of 0.0066±0.0005 μM.

### Example 3—Assessing the Oncolytic Efficacy of QTX125-Containing Combination Therapies

In order to assess the oncolytic efficacy of QTX125-containing combination therapies, monotherapy EC₂₅, EC₅₀ and EC₇₅ doses for sorafenib, gemcitabine and bortezomib were calculated (as per Experiment 3) and administered to wells at these fixed concentrations. QTX125 was co-administered at a range of concentrations, in order to plot a dose-response curve such that the EC₅₀ dose of QTX125 could be calculated in the presence of sorafenib/gemcitabine/bortezomib. QTX125 was administered to cells at a starting high-dose, and a 1:1 dilution was completed thereafter. Cell line-specific dosing regimens for QTX125 and sorafenib, gemcitabine and bortezomib are provided in Tables 4.1, 4.2 and 4.3. In this part of the study, only the hexosaminidase assay was employed to assess cell viability following treatment with QTX125-containing combination therapies.

TABLE 4.1
Dosing regimen used to assess the oncolytic efficacy of QTX125-
containing combination therapies in HCT-116 and MiaPaCa-2 cells.
Values provided are concentrations in micromolar (μM).

| | HCT-116 | | | MiaPaCa-2 | | |
|---|---|---|---|---|---|---|
| | EC₂₅ | EC₅₀ | EC₇₅ | EC₂₅ | EC₅₀ | EC₇₅ |
| Sorafenib | 2 | 3.6 | 6.5 | 4.2 | 6.8 | 10.3 |
| Gemcitabine | 0.001 | 0.01 | 0.1 | 0.01 | 0.1 | 1 |
| Bortezomib | 0.005 | 0.007 | 0.01 | 0.007 | 0.009 | 0.014 |

| | | |
|---|---|---|
| QTX125 | 1 (and diluted 1:1) | 10 (and diluted 1:1) |

TABLE 4.2
Dosing regimen used to assess the oncolytic efficacy of QTX125-
containing combination therapies in Hep-G2 and SK-Ov-3 cells.
Values provided are concentrations in micromolar (μM).

| | Hep-G2 | | | SK-Ov-3 | | |
|---|---|---|---|---|---|---|
| | EC₂₅ | EC₅₀ | EC₇₅ | EC₂₅ | EC₅₀ | EC₇₅ |
| Sorafenib | 0.9 | 2.5 | 6.4 | 9.1 | 12.7 | 21.6 |
| Gemcitabine | 1 | 10 | 100 | 0.1 | 0.25 | 0.5 |
| Bortezomib | 0.014 | 0.016 | 0.026 | 0.006 | 0.007 | 0.012 |

| | | |
|---|---|---|
| QTX125 | 25 (and diluted 1:1) | 10 (and diluted 1:1) |

TABLE 4.3
Dosing regimen used to assess the oncolytic efficacy of
QTX125-containing combination therapies in Panc-1 cells.
Values provided are concentrations in micromolar (μM).

| | Panc-1 | | |
|---|---|---|---|
| | EC₂₅ | EC₅₀ | EC₇₅ |
| Sorafenib | 4.1 | 7.4 | 15 |
| Gemcitabine | 1 | 10 | 100 |
| Bortezomib | 0.007 | 0.009 | 0.015 |

| | |
|---|---|
| QTX125 | 100 (and diluted 1:1) |

It should be noted that it was not possible to determine the appropriate concentrations for gemcitabine in all cell lines tested. In cells resistant to gemcitabine, nominal concentrations were used in place of EC₂₅, EC₅₀ and EC₇₅ doses (e.g., nominal doses of 1, 10 and 100 UM were used in Panc-1 and Hep-G2 cells).

Data demonstrating the oncolytic efficacy of QTX125-containing combination therapies is provided below. Combination indices (Cls) were calculated as described in Example 1, to indicate whether QTX125-containing combination therapies achieve a synergistic effect (decreased EC₅₀), an additive effect (no displacement) or an antagonistic effect (increased EC₅₀) in mediating cell killing as compared to QTX125 monotherapy.

The following index is provided to aid navigation of the data presented in Tables 5.1.1 to 5.5.3:
5.1.x—Assessment of HCT-116 cells
5.2.x—Assessment of MiaPaCa-2 cells
5.3.x—Assessment of Hep-G2 cells
5.4.x—Assessment of SK-Ov-3 cells
5.5.x—Assessment of Panc-1 cells
5.x. 1—Assessment of QTX125/sorafenib combination therapy
5.x.2—Assessment of QTX125/gemcitabine combination therapy
5.x.3—Assessment of QTX125/bortezomib combination therapy

TABLE 5.1.1.
Assessment of the oncolytic efficacy of QTX125/sorafenib
combination therapy in HCT-116 cells. Values provided
are concentrations expressed in nanomolar (nM) or combination
indices (CIs) expressed in arbitrary units.

Sorafenib

| | | — | EC₂₅ | EC₅₀ | EC₇₅ |
|---|---|---|---|---|---|
| QTX125 | EC₅₀ | 2590 | 2346 | 2210 | 2258 |
| | C.I. | — | 0.9 | 0.9 | 0.9 |

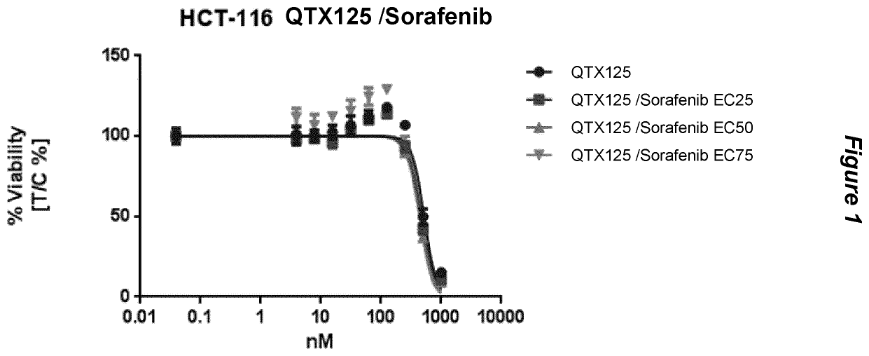

In HCT-116 colonic cancer cells, QTX125/sorafenib combination therapy was found to achieve a synergistic effect in all conditions tested. The data presented in Table 5.1.1 are visualised as dose-response curves, as shown in FIG. 1.

TABLE 5.1.2.
Assessment of the oncolytic efficacy of QTX125/gemcitabine
combination therapy in HCT-116 cells. Values provided
are concentrations expressed in nanomolar (nM) or combination
indices (CIs) expressed in arbitrary units.

Gemcitabine

| | | — | EC₂₅ | EC₅₀ | EC₇₅ |
|---|---|---|---|---|---|
| QTX125 | EC₅₀ | 305.2 | 322 | — | — |
| | C.I. | — | 1.1 | — | — |

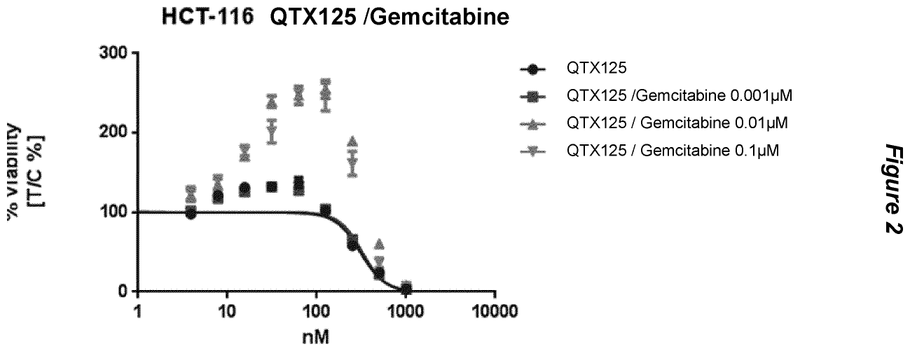

In HCT-116 colonic cancer cells, QTX125/gemcitabine combination therapy was identified as creating an antagonistic effect at the EC₂₅ dose of gemcitabine. The data presented in Table 5.1.2 are visualised as dose-response curves, as shown in FIG. 2. Note that it was not possible to derive a QTX125 dose-response curve from wells dosed with EC₅₀ and EC₇₅ gemcitabine, and therefore Cls could not be calculated.

TABLE 5.1.3.
Assessment of the oncolytic efficacy of QTX125/bortezomib
combination therapy in HCT-116 cells. Values provided
are concentrations expressed in nanomolar (nM) or combination
indices (CIs) expressed in arbitrary units.

Bortezomib

| | | — | EC₂₅ | EC₅₀ | EC₇₅ |
|---|---|---|---|---|---|
| QTX125 | EC₅₀ | 2365 | 2461 | 2536 | 2143 |
| | C.I. | — | 1.0 | 1.1 | 0.9 |

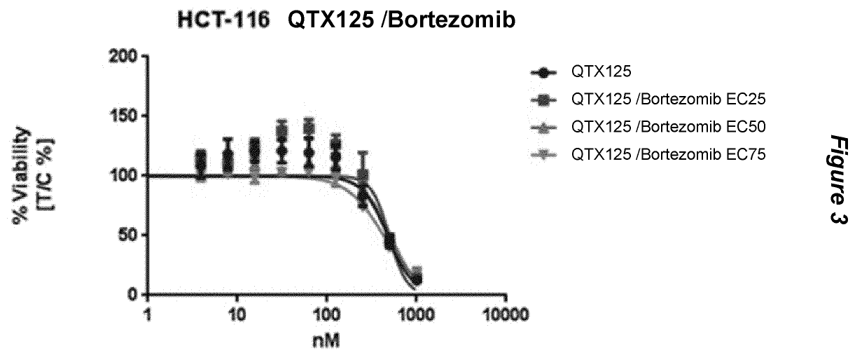

In HCT-116 colonic cancer cells, QTX125/bortezomib combination therapy was found to achieve a synergistic effect at the EC₇₅ dose of bortezomib. An additive effect was noted at the EC₂s dose, and an antagonistic effect was identified at the EC₅₅ dose. The data presented in Table 5.1.3 are visualised as dose-response curves, as shown in FIG. 3.

TABLE 5.2.1.
Assessment of the oncolytic efficacy of QTX125/sorafenib
combination therapy in MiaPaCa-2 cells. Values provided
are concentrations expressed in nanomolar (nM) or combination
indices (CIs) expressed in arbitrary units.

Sorafenib

| | | — | EC₂₅ | EC₅₀ | EC₇₅ |
|---|---|---|---|---|---|
| QTX125 | EC₅₀ | 214 | 258 | 279 | 260 |
| | C.I. | — | 1.2 | 1.3 | 1.2 |

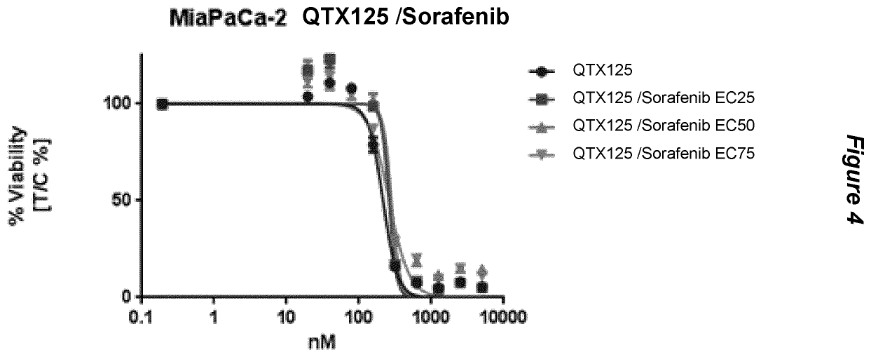

In MiaPaCa-2 pancreatic cancer cells, QTX125/sorafenib combination therapy was found to create an antagonistic effect in all conditions tested. The data presented in Table 5.2.1 are visualised as dose-response curves, as shown in FIG. 4.

TABLE 5.2.2.
Assessment of the oncolytic efficacy of QTX125/gemcitabine
combination therapy in MiaPaCa-2 cells. Values provided
are concentrations expressed in nanomolar (nM) or combination
indices (CIs) expressed in arbitrary units.

Gemcitabine

| | | — | EC₂₅ | EC₅₀ | EC₇₅ |
|---|---|---|---|---|---|
| QTX125 | EC₅₀ | 145.8 | 132.6 | 111.6 | 45.62 |
| | C.I. | — | 0.9 | 0.8 | 0.3 |

In MiaPaCa-2 pancreatic cancer cells, QTX125/gemcitabine combination therapy was found achieve a synergistic effect in all conditions tested. The data presented in Table 5.2.2 are visualised as dose-response curves, as shown in FIG. 4.

TABLE 5.2.3.
Assessment of the oncolytic efficacy of QTX125/bortezomib
combination therapy in MiaPaCa-2 cells. Values provided
are concentrations expressed in nanomolar (nM) or combination
indices (CIs) expressed in arbitrary units.

Bortezomib

| | | — | EC₂₅ | EC₅₀ | EC₇₅ |
|---|---|---|---|---|---|
| QTX125 | EC₅₀ | 230.4 | 239.1 | 238.7 | 245.9 |
| | C.I. | — | 1.0 | 1.0 | 1.1 |

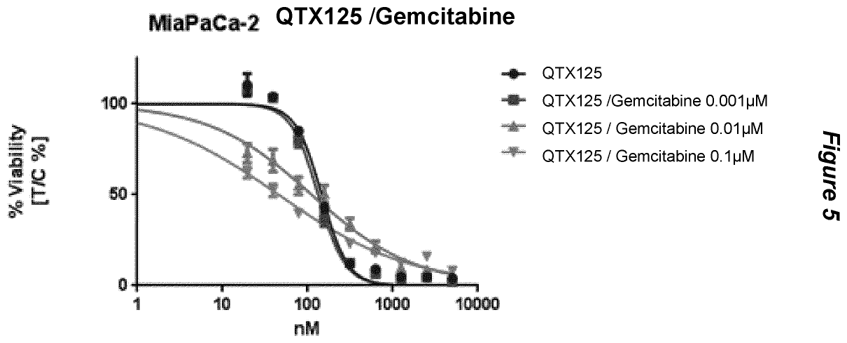

In MiaPaCa-2 pancreatic cancer cells, QTX125/bortezomib combination therapy was found to create an antagonistic effect at the EC₇₅ dose of bortezomib. An additive effect was noted at the EC₂₅ and EC₅₀ doses. The data presented in Table 5.2.3 are visualised as dose-response curves, as shown in FIG. 5.

TABLE 5.3.1.
Assessment of the oncolytic efficacy of QTX125/sorafenib
combination therapy in Hep-G2 cells. Values provided
are concentrations expressed in nanomolar (nM) or combination
indices (CIs) expressed in arbitrary units.

Sorafenib

| | | — | EC₂₅ | EC₅₀ | EC₇₅ |
|---|---|---|---|---|---|
| QTX125 | EC₅₀ | 189.4 | 87.87 | 74.9 | 87.17 |
| | C.I | — | 0.5 | 0.4 | 0.5 |

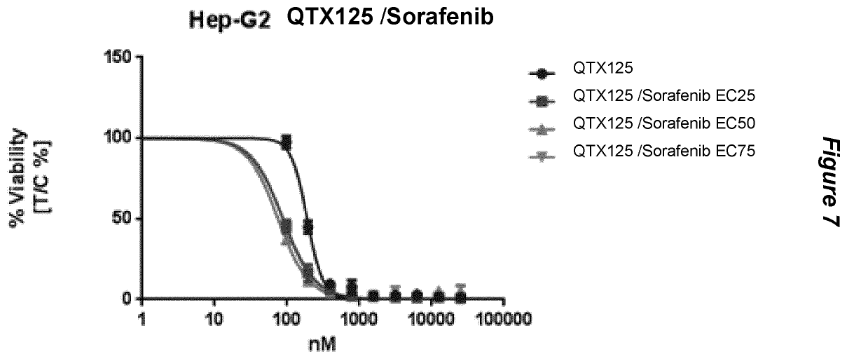

In Hep-G2 hepatic cancer cells, QTX125/sorafenib combination therapy was found to achieve a synergistic effect in all conditions tested. The data presented in Table 5.3.1 are visualised as dose-response curves, as shown in FIG. 7.

TABLE 5.3.2.
Assessment of the oncolytic efficacy of QTX125/gemcitabine
combination therapy in Hep-G2 cells. Values provided
are concentrations expressed in nanomolar (nM) or combination
indices (CIs) expressed in arbitrary units.

Gemcitabine

| | | — | EC₂₅ | EC₅₀ | EC₇₅ |
|---|---|---|---|---|---|
| QTX125 | EC₅₀ | 1196 | 743.9 | 198 | 56.07 |
| | C.I | — | 0.6 | 0.2 | 0.05 |

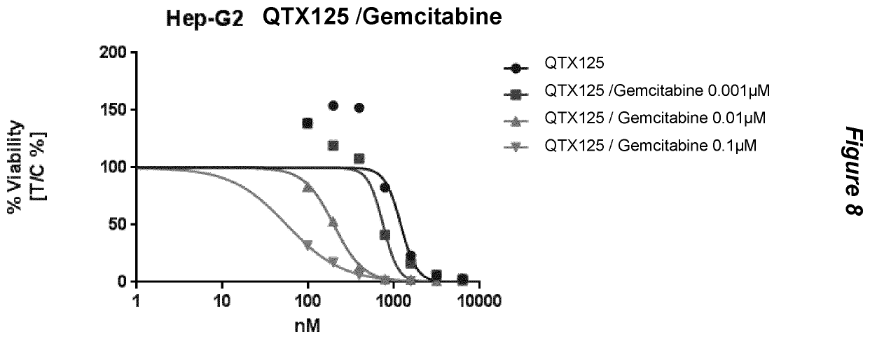

In Hep-G2 hepatic cancer cells, QTX125/gemcitabine combination therapy was found to achieve a synergistic effect in all conditions tested. QTX125/gemcitabine combination therapy was particularly effective at mediating cell killing at the EC₇₅ dose of gemcitabine. The data presented in Table 5.3.2 are visualised as dose response curves, as shown in FIG. 8.

TABLE 5.3.3.
Assessment of the oncolytic efficacy of QTX125/bortezomib
combination therapy in Hep-G2 cells. Values provided
are concentrations expressed in nanomolar (nM) or combination
indices (CIs) expressed in arbitrary units.

Bortezomib

| | | — | EC₂₅ | EC₅₀ | EC₇₅ |
|---|---|---|---|---|---|
| QTX125 | EC₅₀ | 544 | 237 | 128 | 91 |
| | C.I | — | 0.4 | 0.2 | 0.2 |

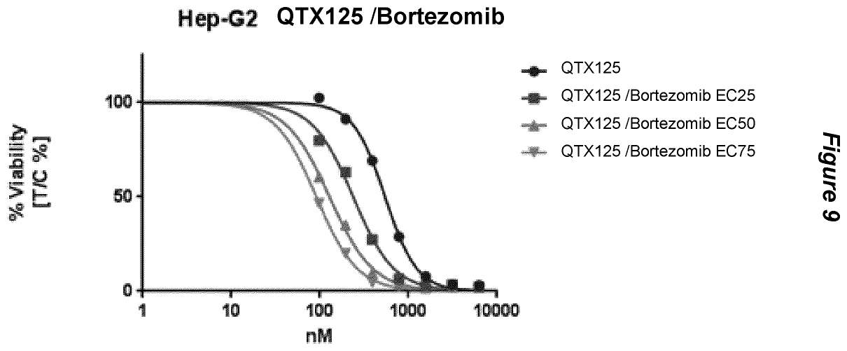

In Hep-G2 hepatic cancer cells, QTX125/bortezomib combination therapy was found to achieve a synergistic effect in all conditions tested. The data presented in Table 5.3.3 are visualised as dose-response curves, as shown in FIG. 9.

TABLE 5.4.1.
Assessment of the oncolytic efficacy of QTX125/sorafenib
combination therapy in SK-Ov-3 cells. Values provided
are concentrations expressed in nanomolar (nM) or combination
indices (CIs) expressed in arbitrary units.

Sorafenib

| | | — | EC₂₅ | EC₅₀ | EC₇₅ |
|---|---|---|---|---|---|
| QTX125 | EC₅₀ | 623 | 389 | 657 | 406 |
| | C.I | — | 0.6 | 1.1 | 0.7 |

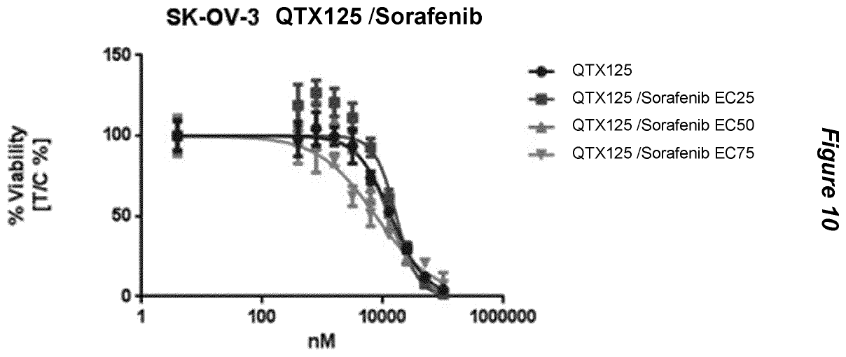

In SK-Ov-3 ovarian cancer cells, QTX 125/sorafenib combination therapy was found to achieve a synergistic effect at the EC₂₅ and EC₇₅ doses. An antagonistic effect was created at the at the EC₅₀ dose. The data presented in Table 5.4.1 are visualised as dose-response curves, as shown in FIG. 10.

TABLE 5.4.2.
Assessment of the oncolytic efficacy of QTX125/gemcitabine
combination therapy in SK-Ov-3 cells. Values provided
are concentrations expressed in nanomolar (nM) or combination
indices (CIs) expressed in arbitrary units.

Gemcitabine

| | | — | EC₂₅ | EC₅₀ | EC₇₅ |
|---|---|---|---|---|---|
| QTX125 | EC₅₀ | 281.9 | 171.3 | 303.1 | 139 |
| | C.I | — | 0.6 | 1.1 | 0.5 |

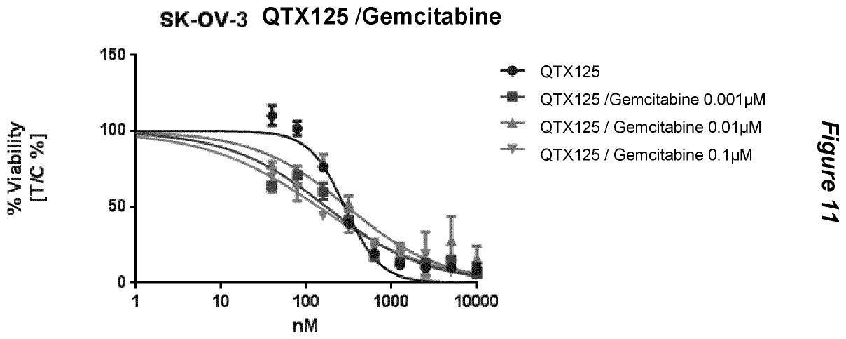

In SK-Ov-3 ovarian cancer cells, QTX125/gemcitabine combination therapy was found to achieve a synergistic effect at the EC₂₅ and EC₇₅ doses. An antagonistic effect was created at the at the EC₅₀ dose. The data presented in Table 5.4.2 are visualised as dose-response curves, as shown in FIG. 11.

TABLE 5.4.3.
Assessment of the oncolytic efficacy of QTX125/bortezomib
combination therapy in SK-Ov-3 cells. Values provided
are concentrations expressed in nanomolar (nM) or combination
indices (CIs) expressed in arbitrary units.

Bortezomib

| | | — | EC₂₅ | EC₅₀ | EC₇₅ |
|---|---|---|---|---|---|
| QTX125 | EC₅₀ | 633 | 532 | 580 | 262 |
| | C.I | — | 0.8 | 0.9 | 0.4 |

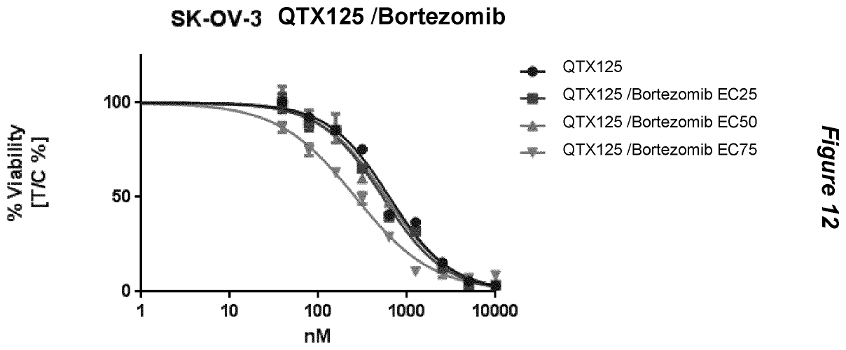

In SK-Ov-3 ovarian cancer cells, QTX125/bortezomib combination therapy was found to achieve a synergistic effect in all conditions tested. The data presented in Table 5.4.3 are visualised as dose-response curves, as shown in FIG. 12.

TABLE 5.5.1.
Assessment of the oncolytic efficacy of QTX125/sorafenib
combination therapy in Panc-1 cells. Values provided
are concentrations expressed in nanomolar (nM) or combination
indices (CIs) expressed in arbitrary units.

| | | Sorafenib | | | |
|---|---|---|---|---|---|
| | | — | EC₂₅ | EC₅₀ | EC₇₅ |
| QTX125 | EC₅₀ | 10152 | 13673 | 142741 | 2982 |
| | C.I | — | 1.3 | 1.4 | 0.3 |

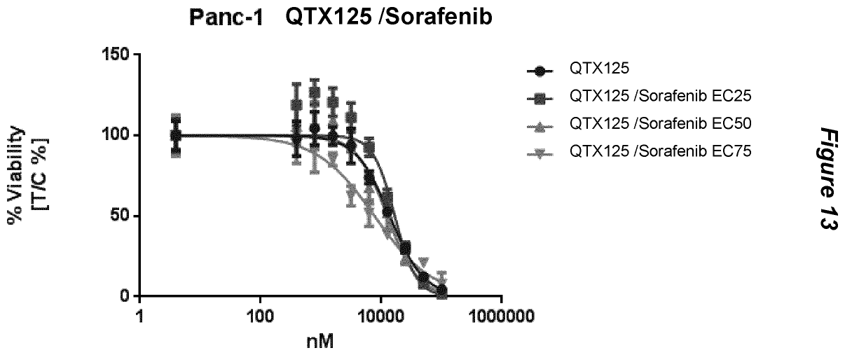

In Panc-1 pancreatic cancer cells, QTX125/sorafenib combination therapy was found to achieve a synergistic effect at the EC₇₅ dose. An antagonistic effect was created at the at the EC₂s and EC₅₀ doses. The data presented in Table 5.5.1 are visualised as dose-response curves, as shown in FIG. 13.

TABLE 5.5.2.
Assessment of the oncolytic efficacy of QTX125/gemcitabine
combination therapy in Panc-1 cells. Values provided
are concentrations expressed in nanomolar (nM) or combination
indices (CIs) expressed in arbitrary units.

Gemcitabine

| | | — | EC₂₅ | EC₅₀ | EC₇₅ |
|---|---|---|---|---|---|
| QTX125 | EC₅₀ | 14749 | 4111 | 2471 | 1940 |
| | C.I | — | 0.3 | 0.2 | 0.1 |

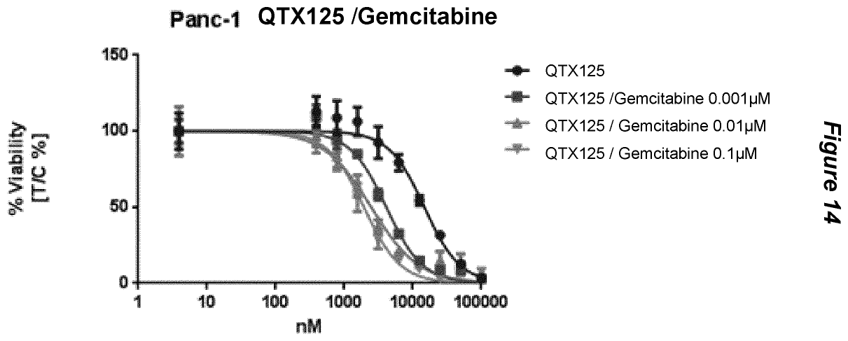

In Panc-1 pancreatic cancer cells, QTX125/gemcitabine combination therapy was found to achieve a synergistic effect in all conditions tested. The data presented in Table 5.5.2 are visualised as dose-response curves, as shown in FIG. 14.

TABLE 5.5.3.
Assessment of the oncolytic efficacy of QTX125/bortezomib
combination therapy in Panc-1 cells. Values provided
are concentrations expressed in nanomolar (nM) or combination
indices (CIs) expressed in arbitrary units.

Bortezomib

| | | — | EC₂₅ | EC₅₀ | EC₇₅ |
|---|---|---|---|---|---|
| QTX125 | EC₅₀ | 18230 | 5034 | 5483 | 1460 |
| | C.I | — | 0.3 | 0.3 | 0.1 |

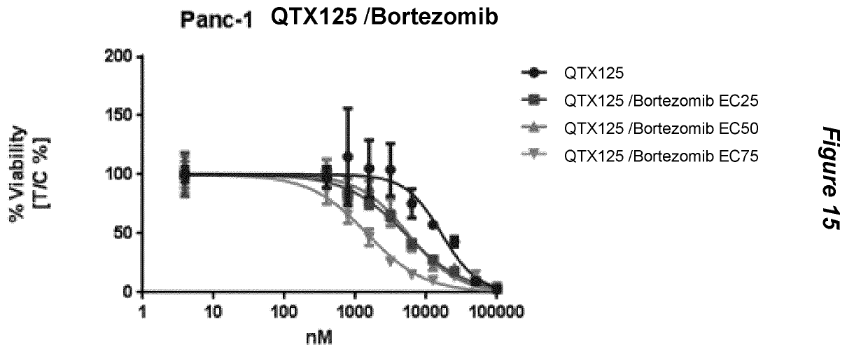

In Panc-1 pancreatic cancer cells, QTX125/bortezomib combination therapy was found to achieve a synergistic effect in all conditions tested. The data presented in Table 5.5.3 are visualised as dose-response curves, as shown in FIG. 15.

### CONCLUSIONS

The aim of this study was to determine the oncolytic efficacy of QTX125 co-administered alongside the chemotherapeutic agents sorafenib, gemcitabine and bortezomib. Efficacy was assessed using five different tumor cell lines, representing colonic, pancreatic, hepatic and ovarian cancers.

EC₅₀ values were identified for all test chemotherapeutic agents in isolation (i.e., as monotherapies), thereby allowing appropriate doses to be identified for use in the combination therapy assays. Dose-response curves were plotted to identify a change in the EC₅₀ of QTX125 co-administered alongside sorafenib, gemcitabine or bortezomib (at EC₂₅, EC₅₀ and EC₇₅ doses). Monotherapy EC₅₀ values were subsequently compared against test EC₅₀ values to calculate combination indices (Cls), indicating whether a given QTX125 combination therapy achieves a synergistic effect (decreased EC₅₀), an additive effect (no displacement), or an antagonistic effect (increased EC₅₀) as compared to QTX125 monotherapy.

The study identified a synergistic effect between QTX125 and bortezomib in all tumor cell lines, with the exception of MiaPaCa-2 pancreatic cancer cells, where an additive/mild antagonistic effect was noted. It should be noted however, that this combination was particularly efficacious in mediating the killing of Panc-1 pancreatic cancer cells.

QTX125/gemcitabine combination therapy similarly achieved a synergistic effect in all tumor cell lines, with the exception of HCT-116 colonic cancer cells, where the combination created a mild antagonistic effect.

The oncolytic efficacy of QTX125/sorafenib combination therapy was more variable however, with a synergistic effect identified in HCT-116, Hep-G2 and Sk-Ov-3 cells. In MiaPaCa-2 cells, the combination created an antagonistic effect. In Panc-1 cells, a synergistic effect was identified at the EC₇₅ dose of sorafenib, whilst an antagonistic effect was created at the EC₂₅ and EC₅₀ doses.

### SUMMARY

In summary, the experimental data provided in this section act to affirm the efficacy and therapeutic utility of the invention. The data successfully demonstrate that QTX125-containing combination therapies achieve synergy, to mediate the efficient killing of colonic, pancreatic, hepatic and ovarian tumor cells in vitro. QTX125-containing combination therapies are therefore likely to be valuable tools for use in the treatment of disease.

### REFERENCES

A number of publications are cited above in order to more fully describe and disclose the invention and the state of the art to which the invention pertains. Full citations for these references are provided below. The entirety of each of these references is incorporated herein.
P. A. Marks et al. in Nature Rev. Cancer 2001, 1, 194;
J. E. Bolden et al. in Nature Rev. Drug Discov. 2006, 5, 769;
P. Gallinari et al. in Cell Res. 2007, 17, 195;
K. B. Glaser in Biochem. Pharmacol. 2007, 74, 659;
L. Pan et al. in Cell. Mol. Immunol. 2007, 4, 337;
M. Haberland et al. in Nature Rev. Genetics 2009, 10, 32;
Y. Zhang et al. in Curr. Med. Chem. 2008, 15, 2840;
S. Ropero, and M. Esteller in Mol. Oncol. 2007, 1, 19
A. G. Kazantsev, and L. M. Thompson in Nature Rev. Drug Discov. 2006, 7, 854
A. Villar-Garea, and M. Esteller in Int. J. Cancer 2004, 112, 171;
T. A. Miller et al. in J. Med. Chem. 2003, 46, 5097;
T. Suzuki and N. Miyata in Curr. Med. Chem. 2005, 12, 2867;
M. Paris et al. in J. Med. Chem. 2008, 51, 1505
J. C. Wong et al. in J. Am. Chem. Soc. 2003, 125, 5586;
G. Estiu et al. in J. Med. Chem. 2008, 51, 2898
K. V. Butler and A. P. Kozikowski in Curr. Pharm. Design 2008, 14, 505;
T. C. Karagiannis and A. El-Osta in Leukemia 2007, 21, 61
WO2011/039353—IkerChem S.L. et al.
WO 2018/087082-Quimatryx S.L.
Perez-Salvia, M. et al in Haematologica 2018; 103: e540
Housman, G et al. in Cancers (Basel) 2014 6 (3): 1769-1792;
Prieto-Callajero, B et al. in Medicine 2020 99 (33): pe21695
Mokhtari, R. et al. in Oncotarget 2017 6 (23): 38025-38043
Citro et al., British Journal of Cancer (2019) 120:658-667
CN 202210325433.9

Handbook of Pharmaceutical Additives, 2nd Edition (eds. M. Ash and I. Ash), 2001 (Synapse Information Resources, Inc., Endicott, New York, USA)

Remington's Pharmaceutical Sciences, 18th edition, Mack Publishing Company, Easton, Pa., 1990

Handbook of Pharmaceutical Excipients, 2nd edition, 1994

For standard molecular biology techniques, see Sambrook, J., Russel, D.W. Molecular Cloning, A Laboratory Manual. 3 ed. 2001, Cold Spring Harbor, New York: Cold Spring Harbor Laboratory Press

1. A method of treatment of a proliferative disorder in a mammalian subject, comprising administering a therapeutically effective amount of a compound of Formula I to a patient in need thereof, 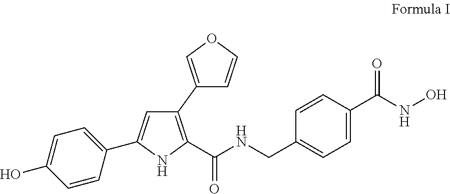 [SMILES: O=C(NO)c1ccc(CNC(=O)c2[nH]c(-c3ccc(O)cc3)cc2-c2ccoc2)cc1]
wherein the method comprises administering a compound of Formula I to the subject simultaneously, sequentially or separately with a second pharmaceutical agent selected from the group consisting of: (i) a protein kinase inhibitor; (ii) a ribonucleotide reductase inhibitor; and (iii) a proteasome inhibitor.

2. The method according to claim 1, wherein the proliferative disorder is a cancer.

3. The method according to claim 2, wherein the cancer comprises a solid tumour.

4. The method according to claim 3, wherein the solid tumour comprises a colorectal tumour, a pancreatic tumour, a hepatic tumour or an ovarian tumour.

5. The method according to claim 1, wherein the second pharmaceutical agent is a protein kinase inhibitor and wherein the protein kinase inhibitor is sorafenib.

6. The method according claim 5, wherein the method is a method of treating colorectal, pancreatic, hepatic or ovarian cancer.

7. The method according claim 5, wherein the method is a method of treating colorectal or hepatic cancer.

8. The method according to claim 1, wherein the second pharmaceutical agent is a ribonucleotide reductase inhibitor, and wherein the ribonucleotide reductase inhibitor is gemcitabine.

9. The method according claim 8, wherein the method is a method of treating pancreatic, hepatic or ovarian cancer.

10. The method according claim 8, wherein the method is a method of treating pancreatic or ovarian cancer.

11. The method according to claim 1, wherein the second pharmaceutical agent is a proteasome inhibitor, and wherein the proteasome inhibitor is bortezomib.

12. The method according claim 11, wherein the method is a method of treating colorectal, pancreatic, hepatic or ovarian cancer.

13. The method according claim 11, wherein the method is a method of treating pancreatic, hepatic or ovarian cancer.

14. The method according any one of claim 11, wherein the method is a method of treating hepatic or ovarian cancer.

15. (canceled)

16. (canceled)

17. (canceled)

18. (canceled)

19. (canceled)

20. A pharmaceutical composition comprising:
a) a first pharmaceutical agent comprising a compound of Formula I or a pharmaceutically acceptable salt thereof 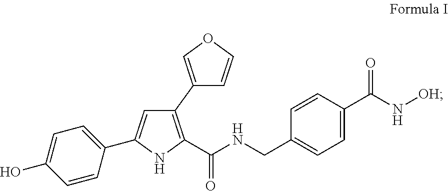 [SMILES: O=C(NO)c1ccc(CNC(=O)c2[nH]c(-c3ccc(O)cc3)cc2-c2ccoc2)cc1]
b) a second pharmaceutical agent selected from the group consisting of: (i) a protein kinase inhibitor; (ii) a ribonucleotide reductase inhibitor; and (iii) a proteasome inhibitor; and
c) a pharmaceutically acceptable carrier, excipient or diluent.

21. The pharmaceutical composition according to claim 20, wherein the second pharmaceutical agent is selected from the group consisting of: sorafenib, gemcitabine and bortezomib.

22. The pharmaceutical composition according to claim 20, wherein the molar ratio of the first pharmaceutical agent to the second pharmaceutical agent is in the range 1:10 to 10:1.

23. (canceled)

24. (canceled)

25. (canceled)

26. (canceled)

27. (canceled)

28. The pharmaceutical composition according to claim 21, wherein the second pharmaceutical agent is sorafenib.

29. The pharmaceutical composition according to claim 21, wherein the second pharmaceutical agent is gemcitabine.

30. The pharmaceutical composition according to claim 21, wherein the second pharmaceutical agent is bortezomib.
